(19) United States
(12) Patent Application Publication
(10) Pub. No.: US 2008/0108871 A1
(43) Pub. Date: May 8, 2008

(54) VACUUM STABILIZED OVERTUBE FOR ENDOSCOPIC SURGERY

(76) Inventor: Catherine J. Mohr, Mountain View, CA (US)

Correspondence Address: PATENT DEPT;INTUITIVE SURGICAL, INC, 1266 KIFER RD, BUILDING 101, SUNNYVALE, CA, 94086, US

(21) Appl. No.: 11/618,374

(22) Filed: Dec. 29, 2006

Related U.S. Application Data
(60) Provisional application No. 60/864,537, filed on Nov. 6, 2006.

(51) Int. Cl. A61B 1/01

(52) U.S. Cl. 600/114

(57) ABSTRACT

An apparatus is provided, including: an overtube having an elongate portion, a distal end, and a proximal end, said overtube defining an instrument lumen extending from the proximal end through the elongate portion to the distal end to permit passage of an instrument; and a suction passage having a proximal end that couples with a vacuum source and a distal end comprising one or more suction ports at the distal end of the overtube. A method of operating a surgical apparatus is provided, including: advancing an overtube into a patient's body, said overtube having an elongate portion, a distal end, and a proximal end, said distal end having one or more suction ports; contacting the distal end of the overtube to a body tissue surface; operating a vacuum source fluidically coupled to the one or more suction ports to adhere the body tissue surface to the distal end of the overtube; and advancing an instrument through an instrument lumen in the overtube.

### CROSS-REFERENCE TO RELATED APPLICATIONS

This application claims the benefit of priority from U.S. provisional patent application Ser. No. 60/864,537, filed on Nov. 6, 2006, entitled “Vacuum Stabilized Overtube for Endoscopic Surgery,” the disclosure of which is incorporated herein in its entirety.

### BACKGROUND

Minimally invasive medical techniques have been used to reduce the amount of extraneous tissue which may be damaged during diagnostic or surgical procedures, thereby reducing patient recovery time, discomfort, and deleterious side effects. Traditional forms of minimally invasive surgery include endoscopy. One of the more common forms of endoscopy is laparoscopy, which is minimally invasive inspection or surgery within the abdominal cavity. In traditional laparoscopic surgery, a patient's abdominal cavity is insufflated with gas and cannula sleeves are passed through small (approximately 5 to 12 mm) incisions in the patient's abdominal wall to provide entry ports through which laparoscopic surgical instruments can be passed in a sealed fashion.

The laparoscopic surgical instruments generally include a laparoscope for viewing the surgical field and working tools with various end effectors. Typical surgical end effectors include clamps, graspers, scissors, staplers, electrocautery devices, suction/irrigation devices, and needle holders, for example. The working tools are similar to those used in conventional (open) surgery, except that the working end or end effector of each tool is separated from its handle by an approximately 12-inch long extension tube, for example, so as to permit the operator to introduce the end effector to the surgical site and to control movement of the end effector relative to the surgical site from outside a patient's body.

In contrast to laparoscopy, flexible endoscopy is a procedure in which a flexible endoscope is inserted into a natural orifice such as the mouth or anus to diagnose and treat medical conditions inside the upper gastrointestinal tract (esophagus, stomach and duodenum) or the lower gastrointestinal tract (rectum, colon and cecum), respectively. The scope will typically be 0.5 to 1 meter long and have a flexible (steerable) tip with a camera and one or two “working channels” through which a flexible instrument may be inserted. These instruments have various end effectors including biopsy forceps, electrocautery needles, suction/irrigation devices, and snares, for example. The instruments available for flexible endoscopy are limited in their capability, so traditionally the medical procedures have been constrained to being performed entirely within the lumen of the GI tract, and have consisted of polyp removal, ablation of vessels, or mucosal resections, for example.

More recently, endoluminal procedures have been used in which the flexible endoscope is inserted into a natural orifice to treat medical conditions outside of the lumen of the gastrointestinal tract. This is achieved by using the instruments to make an incision in the wall of the stomach, for example, and passing the tip of the flexible endoscope into the peritoneal (abdominal) cavity without any incision in the abdominal wall or cutaneous tissue. This allows intraabdominal surgery to be performed entirely through natural orifices.

The introduction of a flexible endoscope through the GI lumen into the abdominal cavity presents significant challenges in controlling contamination, as any perforation made in the wall of the GI tract risks introducing lumen contents such as food, waste products, and intestinal secretions, for example, into the abdominal cavity. In addition, holes made in the wall of the GI tract must be reliably closed at the end of the procedure to prevent post treatment leaks.

It is desirable to provide improved systems and methods for performing trans-luminal endoscopic procedures.

### SUMMARY

An “overtube” comprising a passive flexible tube may be inserted through a natural orifice to provide a passage through which a flexible endoscope, or other flexible trans-luminal endoscopic device, may be passed. This overtube would allow the passage to the operative site to be isolated from the contents of the lumen, and would make insertion, removal, and reinsertion of the endoscopic device simpler to perform. A method is provided for attaching the overtube to the lumen wall through which a flexible endoscope, or other flexible trans-luminal endoscopic device, will be passed. This method prevents the lumen contents from leaking through the incision made into the lumen wall, and from leaking back into the inside of the overtube, thereby contaminating the passageway.

In accordance with exemplary embodiments, an apparatus is provided, including: an overtube having an elongate portion, a distal end, and a proximal end, said overtube defining an instrument lumen extending from the proximal end through the elongate portion to the distal end to permit passage of an instrument; and a suction passage having a proximal end that couples with a vacuum source and a distal end comprising one or more suction ports at the distal end of the overtube.

In accordance with other embodiments, a method of operating a surgical apparatus is provided, including: advancing an overtube into a patient's body, said overtube having an elongate portion, a distal end, and a proximal end, said distal end having one or more suction ports; contacting the distal end of the overtube to a body tissue surface; operating a vacuum source fluidically coupled to the one or more suction ports to adhere the body tissue surface to the distal end of the overtube; and advancing an instrument through an instrument lumen in the overtube.

Other features and aspects of the invention will become apparent from the following detailed description, taken in conjunction with the accompanying drawings which illustrate, by way of example, the features in accordance with embodiments of the invention. The summary is not intended to limit the scope of the invention, which is defined solely by the claims attached hereto.

### DESCRIPTION OF THE DRAWINGS

FIG. 1 shows a surgical assembly.

FIGS. 2A-2C are various views of the distal end of an overtube.

FIG. 3 illustrates an example natural orifice deployment of the overtube in a patient.

FIG. 4 illustrates an overtube including an auxiliary passage for performing additional procedures.

FIGS. 5A-5C illustrate an overtube having a clamp.

FIG. 6 illustrates an embodiment in which an overtube is utilized to attached to the exterior wall of a hollow viscus.

FIGS. 7A-7B illustrate perspective and cross-sectional views, respectively, of the distal end of an overtube.

FIG. 8 illustrates an overtube in accordance with another embodiment.

FIGS. 9A-9B are perspective and cross-sectional views, respectively, of a manifold for an overtube.

FIGS. 10A-10B are perspective and cross-sectional views, respectively, of another embodiment of a manifold for an overtube.

### DETAILED DESCRIPTION

In the following description, reference is made to the accompanying drawings which illustrate several embodiments of the present invention. It is understood that other embodiments may be utilized and mechanical, compositional, structural, electrical, and operational changes may be made without departing from the spirit and scope of the present disclosure. The following detailed description is not to be taken in a limiting sense, and the scope of the embodiments of the present invention is defined only by the claims of the issued patent.

The terminology used herein is for the purpose of describing particular embodiments only and is not intended to be limiting of the invention. Spatially relative terms, such as “beneath”, “below”, “lower”, “above”, “upper”, and the like may be used herein for ease of description to describe one element's or feature's relationship to another element(s) or feature(s) as illustrated in the figures. It will be understood that the spatially relative terms are intended to encompass different orientations of the device in use or operation in addition to the orientation depicted in the figures. For example, if the device in the figures is turned over, elements described as “below” or “beneath” other elements or features would then be oriented “above” the other elements or features. Thus, the exemplary term “below” can encompass both an orientation of above and below. The device may be otherwise oriented (rotated 90 degrees or at other orientations) and the spatially relative descriptors used herein interpreted accordingly.

As used herein, the singular forms “a”, “an”, and “the” are intended to include the plural forms as well, unless the context indicates otherwise. It will be further understood that the terms “comprises” and/or “comprising” specify the presence of stated features, steps, operations, elements, and/or components, but do not preclude the presence or addition of one or more other features, steps, operations, elements, components, and/or groups thereof.

Described herein are embodiments of a system, apparatus, and method for performing minimally-invasive surgical procedures on a patient.

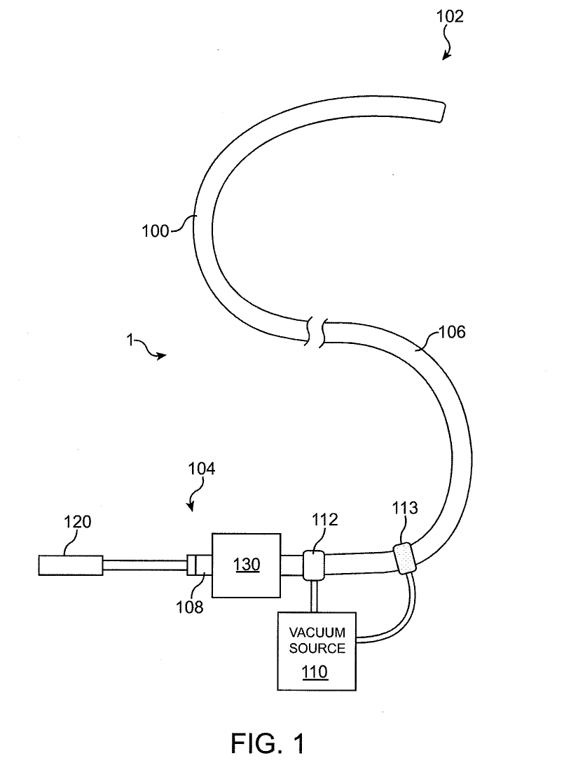

Referring to FIG. 1, a surgical assembly 1 is illustrated according to an embodiment of the present invention. As shown in FIG. 1, the assembly 1 comprises a vacuum stabilized overtube 100 coupled to a vacuum source 110. The overtube 100 comprises an elongate portion 106 having a distal end 102 and a proximal end 104.

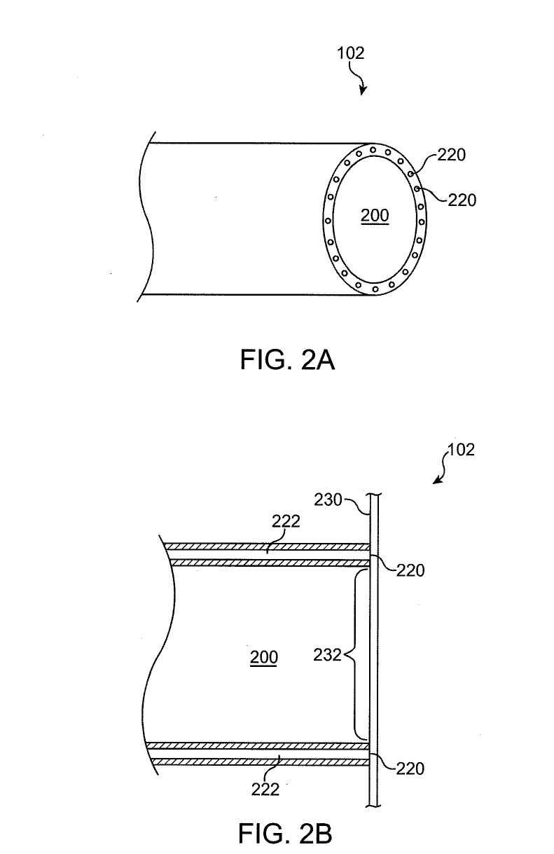

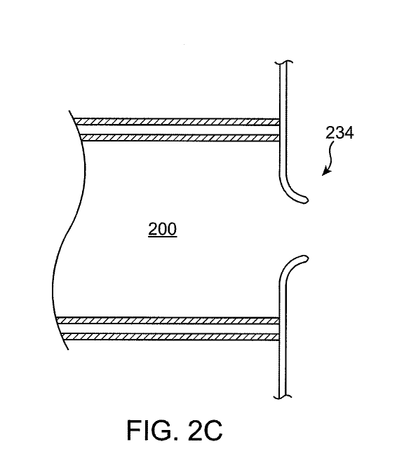

FIGS. 2A-2B are perspective and cross-sectional views, respectively, of the distal end 102 of the overtube 100. The overtube 100 defines an instrument lumen 200 which extends through the overtube 100 from the proximal end 104 to the distal end 102 to permit passage of an instrument 120 through the overtube 100. The overtube 100 further comprises a suction passage 222 which has a proximal end for coupling with vacuum source 110 and a distal end comprising one or more suction ports 220 at the distal end 102 of the overtube 100.

The overtube 100 may, in various embodiments, be formed out of any of a variety of materials suitable for surgical use and may be provided with any of variety of stiffnesses. For example, the overtube 100 may comprise a flexible material, may comprise a substantially rigid material, or may comprise a combination of one or more substantially rigid portions and one or more flexible portions to provide a bendable structure. The overtube 100 may be formed out of a soft flexible material such as polyurethane or polyvinyl chloride. However, any material having comparable physical characteristics may be used. The cross-sectional shape of the overtube 100 may also vary. In the illustrated embodiment, the overtube 100 has a substantially circular cross-sectional shape and is made out of polyurethane. In other embodiments, other cross-sectional shapes may be used, such as, e.g., oval, rectangular, triangular, etc., depending on the application.

In the illustrated embodiment, the suction passage 222 comprises a plurality of vacuum lumens within the wall of overtube 100, with each vacuum lumen terminating at one suction port 220, and all of the vacuum lumens being coupled to the vacuum source 110 via a manifold 112 (FIG. 1). The manifold 112 distributes the vacuum pressure from the vacuum source 110 to the plurality of vacuum lumens.

In the illustrated embodiment, the distal end 102 of the overtube 100 comprises a circular edge, and the suction ports 220 comprise a plurality of ports 220 substantially equally spaced around the circular edge such that when the distal end 102 is pressed against tissue surface 230, the tissue surface 230 will form a seal over the ports 220. The vacuum source 110 may be operated to create a vacuum pressure in the suction passage 220, thereby creating a suction force onto the tissue surface 230 sealing the ports 220. As a result of this suction force, the distal end 102 of the overtube 100 will be attached to the tissue surface 230. If the vacuum pressure is discontinued, the tissue surface 230 will be released and the distal end 102 will no longer be attached to the tissue. Accordingly, by controllably providing a suction at the distal end 102, the overtube 100 can be releasably attached to body tissue surface 230. This body tissue surface 230 may comprise the wall of a lumen or other tissue within the body.

Once the distal end 102 is attached to the tissue surface 230, a containment region 232 of tissue is contained within the walls of the overtube 100. A surgical instrument 120 (FIG. 1) may be inserted through the instrument lumen 200 to carry a suction/irrigation tool to the containment region 232 for washing of the site, or a cutting tool, such as a scissor or other blade, to the containment region 232 for performing surgical procedures. The cutting tool, which is provided at a distal end of the instrument 120, may be used to create an incision 234 in the containment region 232, as shown in FIG. 2C. This incision 234 can then be used to permit access to target surgical site beyond the lumen wall tissue surface 230.

Because the circular edge of the overtube 100 forms an annular seal that surrounds the containment region 232 and attaches the distal end 102 to the tissue surface 230, the position of the incision 234 relative to the distal end 102 of the overtube 100 may be maintained for extended durations so as to enable a surgeon to perform multiple procedures and/or pass multiple instruments through the instrument lumen 200. Multiple instruments may be passed through the instrument lumen 200 to access the target tissue without the necessity of finding the site of the incision 234 each time an instrument is withdrawn and a new instrument inserted. Thus, when the overtube 100 is attached to tissue surface 230 it may be used as a guide for repeated insertion of instruments through a single, minimally invasive surgical incision 234.

The overtube 100 also provides a physical barrier between the instruments utilized by the operator and the patient body environment along the path between the insertion point of the overtube 100 and the containment region 232. The overtube 100 may protect the instruments from the flora, fauna, and other substances that may be encountered in a patient's body. In addition, the overtube 100 may protect the tissue along the path from damage caused by the repeated insertion and withdrawal of instruments. Furthermore, the overtube 100 may provide a physical barrier between the body region through which the overtube 100 is inserted and the body region in which the target tissue is located. The incision in the tissue created to provide access to the target tissue is exposed only to the instrument lumen 200 and not the body lumen.

Various embodiments may be utilized to perform a variety of minimally invasive surgical procedures, including inspection of tissue for diagnosis and patient treatment. This treatment may include, e.g., resective therapies, such as appendectomy, cholecystectomy, and splenectomy or purely modification therapies such as lysis of adhesions, tubal ligations or gastric fundoplication. The overtube 100 may be inserted into a variety of locations within the patient's anatomy by varying the point along the gastrointestinal lumen (or other body passage) at which the end is attached.

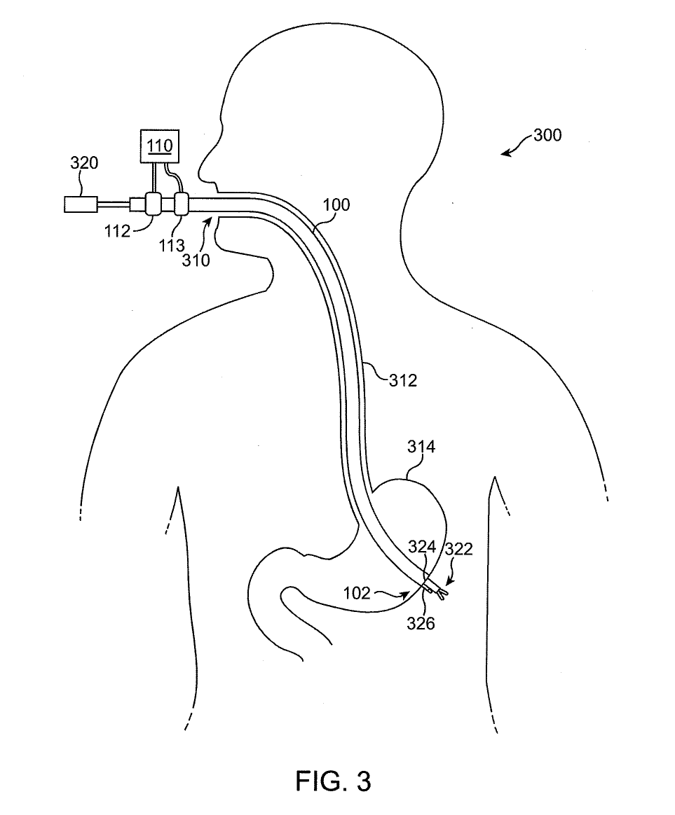

FIG. 3 illustrates an example natural orifice deployment of the overtube 100 in a patient 300. In this example, the overtube 100 is inserted through the patient's mouth 310 and esophagus 312, and into the stomach 314. The distal end 102 of the overtube 100 is then pressed against the interior wall of the stomach 314 and a suction force provided by the vacuum source 110 to attach the distal end 102 to the stomach wall. A surgical instrument 320 having a cutting tool 322 provided at the working end of the instrument 320 is inserted into the overtube 100. The instrument 320 may further include an imaging device 326, such as a camera or optical fiber, in order to enable the operator to view the operation of the cutting tool 322. The cutting tool 322 can then be used to create an incision in the stomach wall, thereby enabling the operator to access the peritoneal cavity with a variety of instruments passed through the overtube. In other embodiments, multiple tools and/or imaging devices may be provided on a single instrument.

As described above, the overtube 100 may serve as a physical barrier in a variety of ways. For example, the overtube 100 protects both the instruments 320 and the peritoneal cavity from the contents of the stomach 314, including gastric juice and other harmful materials. In addition, the overtube 100 may serve as a guide for the insertion of different types of instruments. In some embodiments, the instruments may be flexible so as to passively follow a curved path through the patient's physiology followed by the overtube 100. In other embodiments, the instruments may be rigid or actively controlled.

In many procedures, insufflation is used to expand a body cavity and increase workroom for the investigative and/or surgical procedure. This insufflation results in a positive pressure of the body cavity relative to the environment surrounding the body. When the tissue wall is pierced by the instrument carried by the overtube 100, it may be desirable to prevent the insufflation gases from leaving the body cavity through the instrument lumen 200. This may be accomplished by providing a valve 108 (FIG. 1) at the proximal end 104 where the instrument is inserted into the overtube 100. This valve 108 provides an airtight seal that enables instruments to be inserted and withdrawn while inhibiting insufflation gas leakage through the overtube 100 and out of the proximal end 104. As a result, the insufflation gases may be retained within the body cavity. Examples of these types of single or multi-leaf elastomeric valves may be found in commercially available trocars such as the Excel trocar by Ethicon, Inc. of Somerville, N.J., and are well known to those skilled in the art.

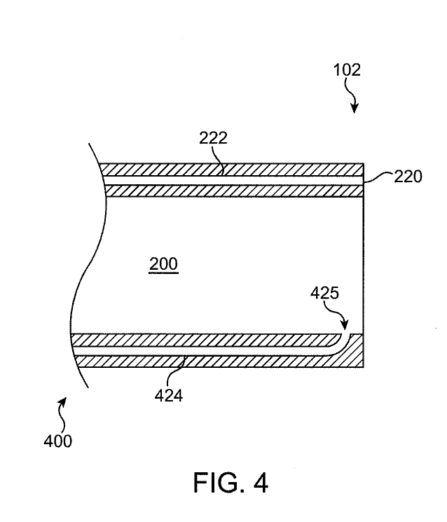

FIG. 4 illustrates an overtube 400 that includes an auxiliary passage 424 for performing additional surgery-related procedures, in accordance with another embodiment. In this embodiment, the auxiliary passage 424 may comprise an evacuation passage having a proximal end (not shown; in one embodiment this proximal end is adjacent the proximal end of overtube 400, which is akin to proximal end 104 of overtube 100 as shown in FIG. 1) for coupling with an evacuation source and a distal end comprising one or more evacuation ports 425 into the instrument lumen 200. The proximal end of the auxiliary passage 424 may comprise, e.g., a second manifold 113 for coupling the evacuation passage 424 to the vacuum source 110 or to a second vacuum source.

The evacuation ports 425 are exposed to instrument lumen 200, in contrast to the suction ports 220, which are sealed against the tissue surface 230. As a result, the evacuation ports 425 are in fluid communication with the target body cavity being accessed by the working end of the surgical instrument. Thus, evacuation passage 424 may be used to provide controlled evacuation of insufflation gas and/or smoke from surgical tool operation.

In other embodiments, the auxiliary passage 424 may be used as an irrigation passage. The irrigation passage may be used to supply fluid to the distal end of the overtube 400. This fluid can be used for a variety of purposes. For example, the fluid may be used as a lavage for cleaning the containment region 232 prior to creating the incision in the tissue surface. The irrigation passage may then be used to drain the fluid from the instrument lumen 200 after the lavage is completed. This lavage and draining can further help to prevent contamination of the target body cavity by contaminating agents contained in the body lumen adjacent tissue surface 230.

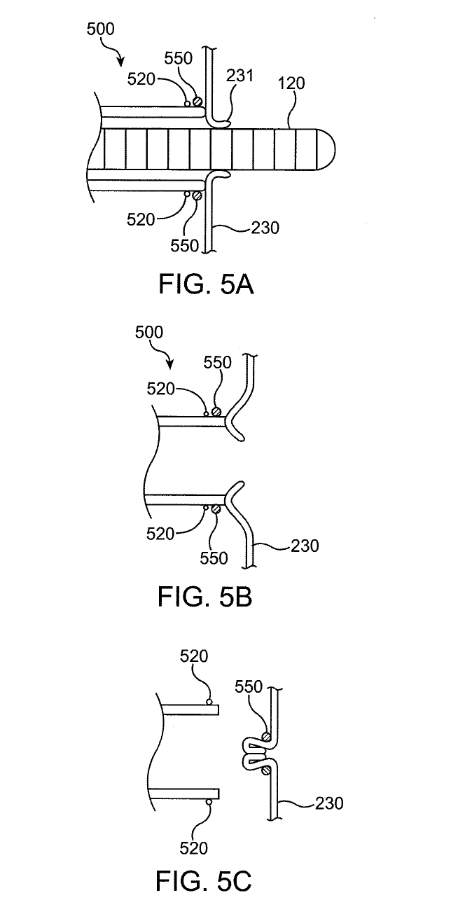

FIGS. 5A-5C illustrate an overtube 500 having a clamp 550 for closing the incision after access to the body cavity is completed. This clamp 550 may comprise, e.g., a circular elastic band, similar to the bands typically used for rubber band ligation.

In FIG. 5A, the incision 231 has been made in the containment region 232 and an instrument 120 has been inserted through the incision 231. After the procedure with the instrument 120 is completed, the instrument 120 is withdrawn. Next, the overtube 500 is slightly withdrawn to raise a portion of tissue surface 230 with respect to the surrounding tissue surface 230, as shown in FIG. 5B. Next, the clamp 550 is pushed off of the distal end 102 of the overtube 500 and around the protruding portion of tissue using, e.g., an actuation member 552. The clamp 550 contracts, thereby clamping the incision closed, as shown in FIG. 5C. Suction is then removed from suction passages 222 so that distal end 102 releases tissue surface 230, leaving clamp 550 in place.

In accordance with various embodiments, the clamp 550 may be used as a primary closure method or as a way of approximating tissue to facilitate subsequent application of clips, sutures, or staples. In some embodiments, the clamp 550 may be dissoluble or bioabsorbable to provide temporary approximation while avoiding necrosis caused by extended clamping.

In accordance with various embodiments, an overtube may be deployed in a variety of locations in the anatomy and using various navigation and steering mechanisms. In some embodiments, the overtube may be inserted into a natural orifice in the body, such as, e.g., oral, rectal, nasal, or vaginal orifices. Alternatively, the overtube may be percutaneously or surgically introduced into another lumen or cavity in the body. Examples of natural lumens include body vessels such as a blood vessel (artery, chamber of the heart or vein), gastrointestinal tract (esophagus, stomach, small and large intestine, cecun and rectum), gynecological tract, or nasopharynx.

In some embodiments, such as when there exists a straight route of access from the natural orifice to the target tissue, the overtube may be rigid. This may be suitable for use in the vaginal fornix or the rectum. In other embodiments, the overtube may be partially or fully flexible. In some embodiments, the assembly 1 may further include a steering control system 120 for steering the overtube using a drivetrain comprising, e.g., guide wires passing through rigid sections forming the overtube 100 so as to navigate through the body lumen. In addition, the overtube may be configured to be locked in a particular shape once the distal end has been attached to the tissue surface. Such steering and locking mechanisms have been used in conventional endoscope systems.

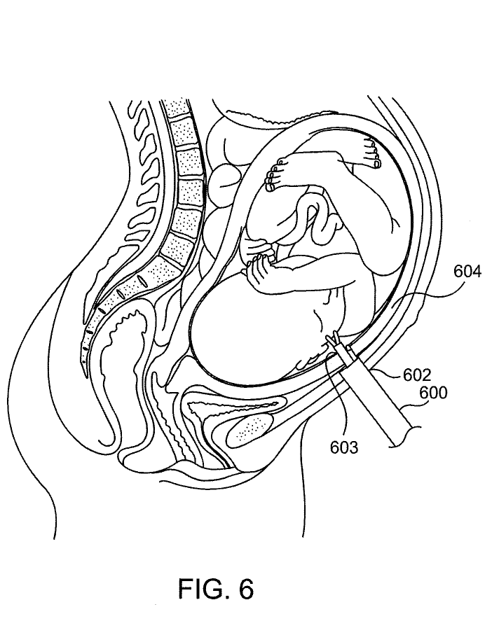

FIG. 6 illustrates one embodiment in which an overtube 600 is utilized to attached to the exterior wall of a hollow viscus within the body. In contrast with natural orifice applications, in this case, the overtube 600 is percutaneously inserted into the body and attached to the outer surface of a hollow viscus, such as the bowel or the gravid uterus. In FIG. 6, a first incision 602 is made to enable the overtube 600 to be inserted percutaneously and attached to the outer wall of the uterus. Next, a cutting instrument is inserted through the instrument lumen in the overtube 600. The cutting instrument is then used to made a second incision 603 in the wall of the uterus 604. Finally, the cutting instrument may be removed and additional surgical and/or diagnostic instruments may be passed through the instrument lumen to gain access to the interior of the uterus 604. The above-described system may be used to perform fetal surgery.

Embodiments of the present invention may provide various advantages not provided by prior art systems. For example, because a non-damaging suction force may be used to attach the distal end of the overtube to the tissue surface, the overtube may be released and easily repositioned in the event that the operator wishes to access a different region of tissue.

In some embodiments, the overtube may be configured for attachment to standard hospital suction units. The dimensions of the suction ports 220 may be selected based on the expected vacuum source so as to provide a sufficient suction force to maintain the attachment to the tissue without causing damage. In some cases, the hospital suction unit may provide 0-1000, 0-600, or 0-250 mbar/hPa vacuum levels.

While the invention has been described in terms of particular embodiments and illustrative figures, those of ordinary skill in the art will recognize that the invention is not limited to the embodiments or figures described. For example, in embodiments described above, the manifold 112 is used to distribute vacuum pressure from the vacuum source 110 to the plurality of vacuum lumens, which extend from the manifold 112 at the proximal end 104 of the overtube 100 to the distal end 102 of the overtube 100. In other embodiments, the manifold 112 may be provided elsewhere along the length of the overtube 100, such as at the distal end 102 or at any intermediate location between the distal end 102 and the vacuum source 110.

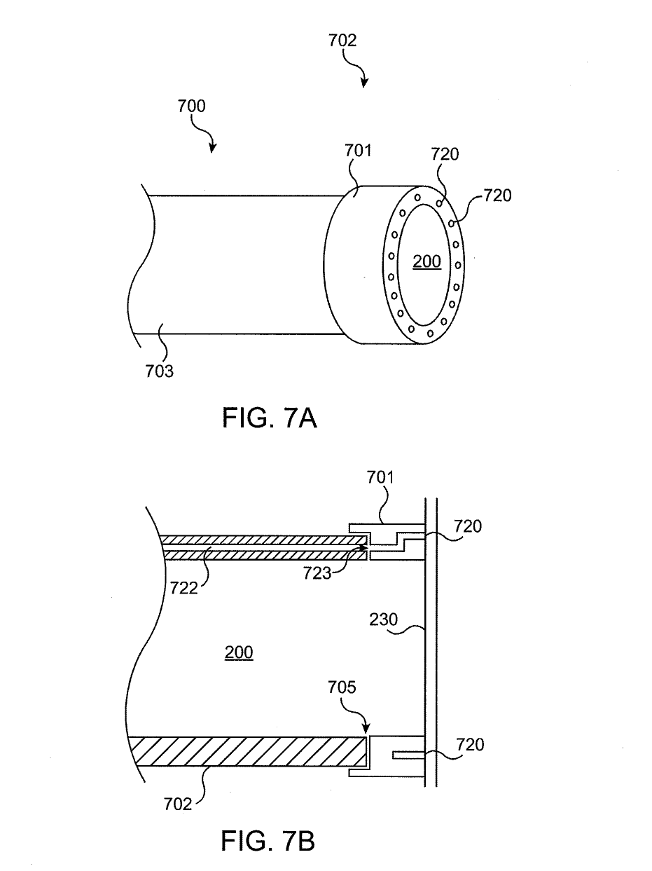

In other embodiments, the overtube 100 may comprise multiple components coupled together to provide the functionality described above. For example, FIGS. 7A-7B illustrate perspective and cross-sectional views, respectively, of the distal end 702 of an overtube 700. Like the overtube 100 described above, the overtube 700 defines an instrument lumen 200 which extends through the overtube 700 from the proximal end (not shown) to the distal end 702 to permit passage of an instrument 120 through the overtube 700. In this embodiment, the overtube 700 comprises an end fitting 701 and a body portion 703. The body portion 703 may be similar to the overtube 100 described above, except that the distal end 705 of the body portion 703 is coupled to the end fitting 701, rather than pressed against the tissue surface. The body portion 703 includes a suction passage 722 coupled to the vacuum source 110. In the illustrated embodiment, the suction passage 722 comprises a single vacuum lumen, which terminates at a corresponding outlet 723 in the end fitting 701. The end fitting 701 includes a manifold for distributing the vacuum pressure from the outlet 723 to a plurality of suction ports 720, which are used to attach the distal end 702 of the overtube 700 to the tissue surface 230.

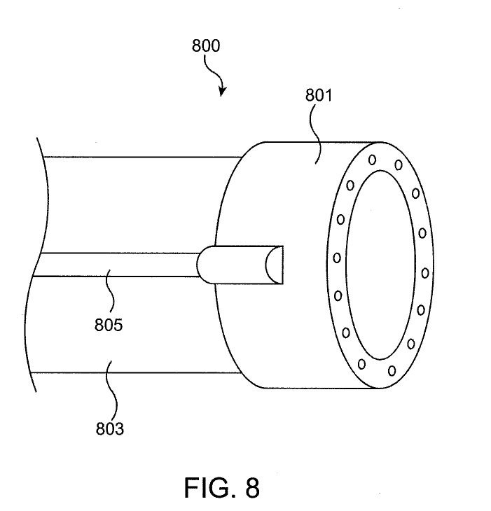

In this embodiment, the suction passage 722 is provided in the outer wall defining the instrument lumen 200. In the embodiment shown in FIG. 8, the overtube 800 comprises a pair of tubes: an instrument tube 803 and a suction tube 805. The instrument tube 803 may be coupled to the suction tube 805 along the length of the overtube 800, or may be coupled at selected points along the length of the overtube 800 (e.g., at the proximal and distal ends). In this case, the suction tube 805 defines suction passage through which the vacuum pressure is applied, and the instrument tube 803 defines the instrument lumen 200 through which the surgical instruments may be passed. It will be understood that in other embodiments, the arrangement of the various components may vary.

In addition, in embodiments described above, a single manifold distributes the vacuum pressure from the vacuum source to the plurality of vacuum lumens. If the vacuum lumens are in fluid communication with each other, then if the vacuum seal is broken between one of the suction ports 220 and the tissue surface, the leakage of air through the broken seal may release the other ports from the tissue surface. Accordingly, it may be desirable for at least some of the ports to be isolated from the other ports. Thus, suction failure of one port will not affect the suction for the isolated ports, thereby maintaining the attachment between the overtube and the tissue surface.

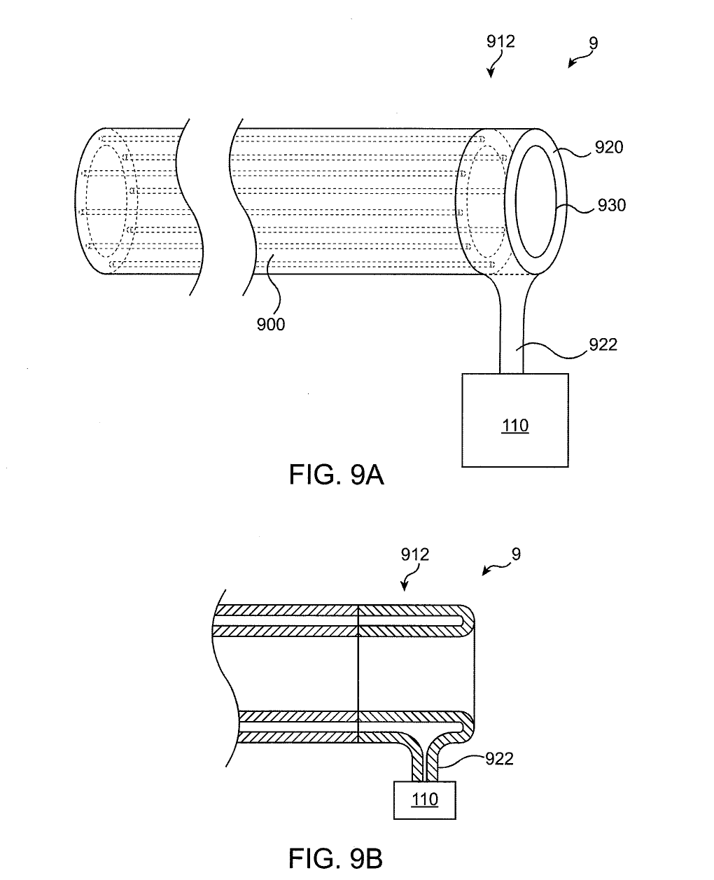

The manifold 112 which couples the vacuum source 110 to the suction passage 222 may be provided in a variety of ways. FIGS. 9A-9B are perspective and cross-sectional views, respectively, of an assembly 9 having a manifold 912, in accordance with one embodiment. In this embodiment, the manifold 912 comprises an end cap 920 that is bonded to the proximal end of the overtube 900. The plurality of vacuum lumens forming the suction passage 222 terminate at the proximal end of the overtube 900 and are in fluid communication with an outlet 922 in the manifold 912. The outlet 922 is in fluid communication with the vacuum source 110. The end cap 920 includes a port 930 through which the various surgical instruments may pass.

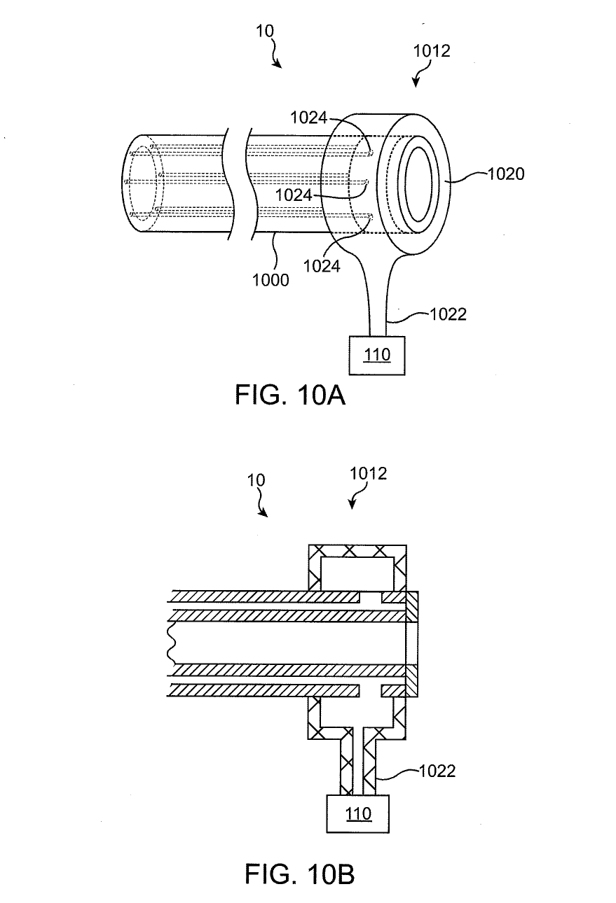

FIGS. 10A-10B are perspective and cross-sectional views, respectively, of an assembly 10 having a manifold 1012, in accordance with another embodiment. In this embodiment, the plurality of vacuum lumens forming the suction passage 222 terminate in a plurality of ports 1024 along the outer wall of the overtube 1000. These ports 1024 may be drilled or formed in the wall of the overtube 1000. The manifold 1012 comprises an annular ring 1020, which is bonded onto the overtube 1000. The manifold 1012 places the ports 1024 in fluid communication with the vacuum source 110 via an outlet 1022. In this embodiment, because the manifold 1012 is operatively coupled to the vacuum lumens of the suction passage 222 via the outer wall of the overtube 1000, it is possible for the overtube 1000 to extend more proximally beyond the manifold 1012.

The shape of the various components described above may vary. For example, the overtube need not have a circular cross-section. In other embodiments, the cross-section may vary. In addition, the profile of the distal edges of the overtubes may vary. In some cases, the edges may be rounded to prevent damage to the adhering tissue.

Therefore, it should be understood that the invention can be practiced with modification and alteration within the spirit and scope of the appended claims. The description is not intended to be exhaustive or to limit the invention to the precise form disclosed. It should be understood that the invention can be practiced with modification and alteration and that the invention be limited only by the claims and the equivalents thereof.

What is claimed is:

1. An apparatus, comprising:
an overtube having an elongate portion, a distal end, and a proximal end, said overtube defining an instrument lumen extending from the proximal end through the elongate portion to the distal end to permit passage of an instrument; and
a suction passage having a proximal end that couples with a vacuum source and a distal end comprising one or more suction ports at the distal end of the overtube.

2. The apparatus of claim 1, wherein:
said one or more suction ports comprise a plurality of suction ports; and
a manifold that provides gaseous communication between the vacuum source and the plurality of suction ports.

3. The apparatus of claim 1, wherein:
the proximal end of the overtube comprises an instrument access port that receives the instrument into the instrument lumen.

4. The apparatus of claim 3, wherein:
said instrument access port comprises a valve that allows passage of an instrument through the instrument access port and substantially prevents gas from exiting the instrument access port.

5. The apparatus of claim 1, further comprising:
an evacuation passage that couples an evacuation source with an evacuation port into the instrument lumen.

6. The apparatus of claim 5, wherein:
said evacuation port is provided adjacent to the distal end of the overtube.

7. The apparatus of claim 1, further comprising:
an irrigation passage having a proximal end for coupling with a fluid source and a distal end comprising one or more irrigation ports into the instrument lumen.

8. The apparatus of claim 7, wherein:
said one or more irrigation ports are provided adjacent to the distal end of the overtube.

9. The apparatus of claim 1, further comprising:
a clamp positioned at the distal end of the overtube, said clamp being deployable to seal an incision created in tissue surrounded by the distal end of the overtube.

10. The apparatus of claim 1, wherein:
said overtube is rigid from the proximal end to the distal end.

11. The apparatus of claim 1, wherein:
said overtube is flexible.

12. The apparatus of claim 11, further comprising:
a steering control system for actuating bending of said overtube.

13. The apparatus of claim 1, wherein:
the instrument comprises a diagnostic instrument.

14. The apparatus of claim 1, wherein:
the instrument comprises a therapeutic instrument.

15. A method operating a surgical apparatus, comprising:
advancing an overtube into a patient's body, said overtube having an elongate portion, a distal end, and a proximal end, said distal end having one or more suction ports;
contacting the distal end of the overtube to a body tissue surface;
operating a vacuum source fluidically coupled to the one or more suction ports to adhere the body tissue surface to the distal end of the overtube; and
advancing an instrument through an instrument lumen in the overtube.

16. The method of claim 15, further comprising:
forming an incision in the body tissue surface; and
advancing the instrument through the opening in the body tissue surface.

17. The method of claim 16, further comprising:
utilizing the instrument to perform a surgical procedure on a target site opposite the body tissue surface from the overtube.

18. The method of claim 15, wherein:
said one or more suction ports comprise a plurality of suction ports; and
said operating the vacuum source comprises coupling the vacuum source to the plurality of suction ports via a manifold that provides gaseous communication between the vacuum source and the plurality of suction ports.

19. The method of claim 15, wherein:
said advancing the instrument through the instrument lumen comprises inserting the instrument into an instrument access port in the proximal end of the overtube.

20. The method of claim 19, wherein:
said instrument access port comprises a valve that allows passage of an instrument through the instrument access port and substantially prevents gas from exiting the instrument access port.

21. The method of claim 15, further comprising:
evacuating a fluid from the instrument lumen via an evacuation port.

22. The method of claim 21, wherein:
said evacuating the fluid comprises evacuating the fluid from the evacuation port provided adjacent to the distal end of the overtube.

23. The method of claim 15, further comprising:
supplying a fluid to one or more irrigation ports in the instrument lumen.

24. The method of claim 23, wherein:
said supplying the fluid to the one or more irrigation ports comprises supplying the fluid to one or more evacuation ports provided adjacent to the distal end of the overtube.

25. The method of claim 15, further comprising:
displaying a clamp positioned at the distal end of the overtube to seal an incision created in tissue surrounded by the distal end of the overtube.

26. The method of claim 15, wherein:
said overtube is rigid from the proximal end to the distal end.

27. The method of claim 15, wherein:
said overtube is flexible.

28. The method of claim 15, further comprising:
utilizing a steering control system to actuate bending of said overtube.

29. The method of claim 15, wherein:
the instrument comprises a diagnostic instrument.

30. The method of claim 15, wherein:
the instrument comprises a therapeutic instrument.
